(19) United States
(12) Patent Application Publication
(10) Pub. No.: US 2008/0215137 A1
(43) Pub. Date: Sep. 4, 2008

(54) THERAPEUTIC DRIVING LAYER FOR A MEDICAL DEVICE

(75) Inventors: Samuel J. EPSTEIN, Watertown, MA (US); Irina OSTROVSKY-DAY, Boston, MA (US); Wendy NAIMARK, Cambridge, MA (US); Anastasia PANOS, Athens (GR)

Correspondence Address: KENYON & KENYON LLP, 1500 K STREET N.W., SUITE 700, WASHINGTON, DC, 20005, US

(73) Assignee: BOSTON SCIENTIFIC SCIMED, INC., MAPLE GROVE, MN (US)

(21) Appl. No.: 11/835,787

(22) Filed: Aug. 8, 2007

Related U.S. Application Data
(63) Continuation-in-part of application No. 10/426,642, filed on Apr. 30, 2003.
Application No. 10/426,642 is a continuation-in-part of application No. 10/989,519, filed on Nov. 17, 2004, now abandoned.

(51) Int. Cl. A61F 2/06

(52) U.S. Cl. 623/1.42; 623/1.46

(57) ABSTRACT

The present invention regards the delivery of therapeutic at a target site. Systems that employ the present invention may employ a medical device sized to be inserted into a target site, a driving layer covering at least a portion of an accessible surface of the medical device, and a therapeutic interfaced with at least a portion of the driving layer. In this system, the driving layer may have a material characteristic that serves to release the therapeutic from the medical device when the medical device is at the target site. Other systems that employ the invention may also have properties that include having a driving layer with a higher solubility than the therapeutic at the target site, a medical device that is hydrophobic while the therapeutic is hydrophilic, and a coating covering at least a portion of the therapeutic.

### CROSS-REFERENCE TO RELATED APPLICATIONS

This application is a continuation-in-part of and claims priority to co-pending U.S. patent application Ser. Nos. 10/426,642 (filed 30 Apr. 2003) and 10/989,519 (filed 17 Nov. 2004), which are both incorporated by reference herein.

### FIELD OF THE INVENTION

The present invention regards the use of a driving material to facilitate the release or delivery of therapeutic from a medical device. More specifically, the present invention regards the use of a driving layer, which may be positioned in contact with a therapeutic and the medical device, the driving layer facilitating the release or delivery of therapeutic from the medical device to a target site.

### BACKGROUND

The delivery of therapeutics to a target site is an often repeated procedure in contemporary medicine. In some instances the therapeutic may be simply injected into the vasculature of a patient in order to reach a target site. In other instances, the delivery of the therapeutic is more focused, being intended to interface with specific target regions or target tissue, whether they be inside or outside the body of a patient. The more focused delivery techniques can be carried out by invasive as well as non-invasive procedures. For instance, an implant or a catheter may each be used to deliver therapeutic to a specific target site such as the hip. There are other methods for therapeutic delivery as well.

When medical implants are employed by a practitioner they may be used to deliver therapeutic to a target site, to reinforce a target site, for both of these reasons, and for other reasons as well. For instance, in addition to delivering therapeutic, an implant may also be used to support collapsed vessels in the vasculature, replace missing tissue or bone throughout the body of a patient, and supplement existing tissue and structures within a patient. These implants may be made with natural, synthetic, metallic or hybrid materials and may be intended to be placed at a target site for both short and prolonged periods of time. During these various implant life cycles, a therapeutic carried by the implant may be delivered prior to placement of the implant, immediately upon placement of the implant, over longer periods of time after the implant is delivered, and in various combinations of these delivery time spans.

### SUMMARY OF THE INVENTION

The present invention regards the delivery of therapeutic at a target site. Quite often this target site will be within the body of a patient. It may, however, be elsewhere. Systems that employ the present invention may employ a medical device sized to be inserted into a target site, a driving layer covering at least a portion of an accessible surface of the medical device, and a therapeutic interfaced with at least a portion of the driving layer. In this system, the driving layer may have a material characteristic that serves to release the therapeutic from the medical device when the medical device is at the target site. Other systems that employ the invention may also have properties that include having a driving layer with a higher solubility than the therapeutic at the target site, a medical device that is hydrophobic while the therapeutic is hydrophilic, and a coating covering at least a portion of the therapeutic. Furthermore, the driving layer may serve as a mechanism to release the therapeutic from the medical device, as a mechanism to retain the therapeutic on the medical device prior to the release of the therapeutic from the medical device, and as a transfection agent. There are further uses and embodiments of the present invention.

### DETAILED DESCRIPTION

The medical devices appropriate for use in the present invention have a surface that is suitable for exposure to body tissue. Suitable medical devices include, but are not limited to, stents, surgical staples, catheters, such as central venous catheters and arterial catheters, guidewires, cannulas, cardiac pacemaker leads or lead tips, cardiac defibrillator leads or lead tips, GDC coils, implantable vascular access ports, blood storage bags, blood tubing, vascular or other grafts, intra-aortic balloon pumps, heart valves, cardiovascular sutures, total artificial hearts and ventricular assist pumps, extra-corporeal devices such as blood oxygenators, blood filters, hemodialysis units, hemoperfusion units, or plasmapheresis units.

Medical devices which are particularly suitable for the present invention include any stent for medical purposes, which are known to the skilled artisan. Suitable stents include, for example, vascular stents such as self-expanding stents and balloon expandable stents. Examples of self-expanding stents are illustrated in U.S. Pat. Nos. 4,655,771 and 4,954,126 issued to Wallsten and U.S. Pat. No. 5,061,275 issued to Wallsten et al. Examples of appropriate balloon-expandable stents are shown in U.S. Pat. No. 5,449,373 issued to Pinchasik et al. The framework of the suitable stents may be formed through various methods as known in the art. The framework may be welded, molded, laser cut, electro-formed, or consist of filaments or fibers which are wound or braided together in order to form a continuous structure.

The medical devices suitable for the present invention may be fabricated from polymeric and/or metallic materials. Suitable polymeric materials include without limitation polyurethane and its copolymers, silicone and its copolymers, ethylene vinyl-acetate, polyethylene terephthalate, thermoplastic elastomers, polyvinyl chloride, polyolefins, cellulosics, polyamides, polyesters, polysulfones, polytetrafluorethylenes, polycarbonates, acrylonitrile butadiene styrene copolymers, acrylics, polylactic acid, polyglycolic acid, polycaprolactone, polylactic acid-polyethylene oxide copolymers, cellulose, collagens, and chitins. Suitable metallic materials include metals and alloys based on titanium (such as nitinol, nickel titanium alloys, thermo-memory alloy materials), stainless steel, tantalum, nickel-chrome, or certain cobalt alloys including cobalt-chromium-nickel alloys such as Elgiloy® and Phynox®. Metallic materials also include clad composite filaments, such as those disclosed in WO 94/16646.

The medical devices of the present invention have a first coating layer and a second coating layer. The first coating layer is disposed on at least a portion of the surface of the medical device. The first coating layer may be biocompatible and/or biodegradable. For example, the first coating layer may include a biological activator such as, but is not limited to, biodegradable polymers, elastomers, and oligomers of such monomers as caprolactone, lactic and glycolic acid, hydroxybutyrate, trimethylene carbonate, amino acids, anhydrides, or any variety of vinyl alcohols, biocompatible salts, proteins such as albumin, collagen, elastin or ECM matrices, dextran, and heparin, ionic and non-ionic surfactants that can be effective transfection agents, cationic lipids, polyethyleneimines, and chitosan.

Generally, the first coating layer comprises a component that both reduces the surface tension of the surface of the medical device while also enhancing the delivery of the therapeutic agent in the second coating layer to body tissue. For example, if the therapeutic agent is a protein, the first coating layer may be a conformation stabilizer such as albumin. When the therapeutic agents are cells, the first coating layer may be an extracellular matrix scaffold. If the therapeutic agent is a gene or genetic material, the first coating layer may be a transfection agent that enhances transfection of genetic material such as plasmid DNA.

In some cases, the first coating layer comprises a transfection agent. Preferably, the transfection agent is biodegradable. The transfection agent is preferably a relatively inexpensive, biocompatible material that promotes the adhesion of the second coating layer to the surface of the medical device, particularly during the coating process. More particularly, the transfection agent reduces the surface tension of the surface of the medical device so that adhesion of the second coating layer is enhanced as compared to when the first coating layer is absent.

The transfection agent also enhances the delivery of the therapeutic agent to the targeted body tissue. Thus, the transfection agent used in the present invention makes it more efficient to apply aqueous solutions or dispersions of therapeutic agents onto a stent while enhancing transfection of the therapeutic agent into the targeted body tissue.

Suitable transfection agents include, but are not limited to, poloxamers, dendrimers, polyvinyl pyrrolidone, liposomes, chitosan, calcium, and glycerol. Preferred transfection agents include, but are not limited to, poloxamers and dendrimers. Poloxamers are surfactants. A poloxamer is a series of copolymers composed of polyoxyethylene and polyoxypropylene blocks. The poloxamers vary in total molecular weight, polyoxypropylene to polyoxyethylene ratio, and surfactant properties.

The second coating layer that is disposed over at least a portion of the first coating layer, can partially or fully cover the first coating layer. The second coating layer comprises a therapeutic agent to be delivered to body tissue.

In certain embodiments, the first coating layer is a driving layer, which comprises a driving material, and the second coating layer comprises a therapeutic agent. The driving material serves to, inter alia, improve the release characteristics of a therapeutic from a medical device. The driving material of the present invention may facilitate the release of therapeutic from the medical device by having a higher solubility than the therapeutic, by having a higher rate of degradation than the therapeutic or by having some other property that results in the therapeutic being more prone to be driven off of the medical device when the medical device is at a target site. Moreover, in some instances not only will the driving layer act to drive or impel the therapeutic off of the medical device, it may also act to prevent the therapeutic from being dissolved in the medical device and to secure the therapeutic to the medical device until such time as the medical device is positioned near a target site.

The driving material of the present invention may comprise a protein that has been positioned onto a surface of a medical implant such as a metal stent while the therapeutic may include strands of DNA. In this example, prior to deployment or placement of the metal stent, the protein can act to affix the DNA to the stent, then, upon deployment, as the protein begins to dissolve, it may now act to release the DNA from the stent. In this example, as well as in others, the driving layer may exhibit other benefits as well. For one, it may serve as a transfection agent improving the delivery across cell membranes of the therapeutic placed on the stent or other medical device. For another, the driving layer may act as a shield between a hydrophillic therapeutic and a hydrophobic metal implant, thereby improving the delivery characteristics of systems employing these materials. There are other benefits and uses of the invention as well.

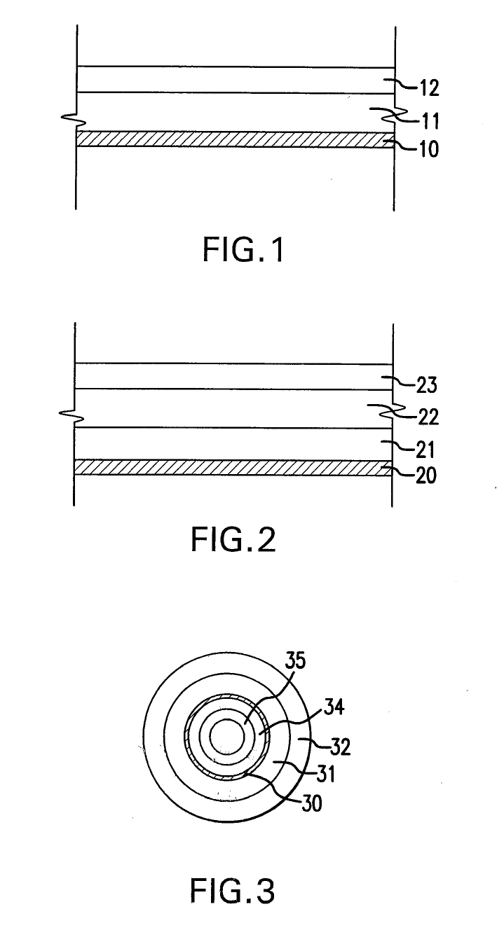

FIG. 1 is a side sectional view of a medical device 10 that contains a driving layer 11 and a therapeutic 12 in accord with the present invention. The medical device 10 employed may be any one of numerous medical devices that may be used to position and deploy therapeutic. This would include balloon catheters, metal stents, and soft or hard tissue implants. Likewise, various materials may comprise the driving layer 11 and the driving layer 11 may coat the entire medical device or only portions of it. In this embodiment the exposed outer surface of the medical device 10 is completely coated with the driving layer 11 while a therapeutic 12 is positioned on top of the driving layer 11. The therapeutic 12 may cover the entire medical device, only the portions covered with the driving layer and other areas as well. In FIG. 1, the therapeutic covers the entire medical device 10.

The driving layer 11 may be more soluble at the environment of the target site than the therapeutic 12. The driving layer 11 may also be more compatible with the medical device than the therapeutic 12. In addition, as mentioned above, the driving layer 11 may even act as a transfection agent for the therapeutic 12 upon the implant's delivery to the target site. In so doing, therapeutic may be more efficiently delivered across cell boundaries at the target site. Still further, the driving layer 11 may also facilitate the release of the therapeutic through the driving layer's own degradation at the target site. In other words, covered by and supporting the therapeutic, as the driving layer degrades at the target site, therapeutic supported by the driving layer would be released from the medical device as a result of the degradation.

The driving layer of the present invention may be applied to the medical device in numerous ways. It may be sprayed onto the medical device, poured over the medical device while in solution, and directly deposited on the medical device to name a few. When the driving layer is sprayed, application systems and methodologies that reduce or eliminate the amount of webbing of the applied driving layer on a medical device are preferred. When the driving layer is poured over the device within a carrier medium, this medium may evaporate away, leaving the driving layer behind.

The therapeutic may, likewise, also be applied to the medical device using various methods and techniques. These would include spray, liquid interface (i.e., pouring the therapeutic over the medical device), and direct deposition. When the therapeutic is poured using an aqueous carrying solution, some of the therapeutic may become embedded in the driving layer as the aqueous carrying solution may displace or erode some of the driving layer. In this instance, now embedded in the driving layer, the therapeutic may be released from the driving layer when the driving layer begins to dissolve or degrade. Preferred methods for applying the therapeutic and the driving layer would reduce waste and overspray of both the therapeutic and the driving layer.

The driving layer 11, which may be highly soluble, highly degradable or both, may be an ionic salt, an ionic surfactant, a non-ionic surfactant, a swellable polymer, a lipid, a polysaccharide, a foaming agent, an inorganic polymer, a block copolymer, a dissolvable polymer (such as Dextran) or any suitable combination or mixture.

In one specific example, a water soluble protein may be used as the driving layer. This layer may serve to first adhere therapeutic to the surface of the medical device and then to provide a complete release of the therapeutic upon dissolving. Thus, in this instance as well as in other examples, is it preferred that the driving layer be more soluble, more degradable or both when compared with the therapeutic that overlies it. Furthermore, in this and the other embodiments it is preferred that the driving layer be bio-compatible. The therapeutic 12 in this and the other embodiments may be DNA, a protein or various cell therapies. Other therapeutics, which are itemized below, may also be suitable.

FIG. 2 is a side view of a medical device 20 in accord with the invention. As in FIG. 1, the medical device 20 in this figure may be chosen from various medical devices including both polymer and non-polymer devices. The medical device 20 in this figure is covered by a driving layer 21, a therapeutic 22, and a coating 23. The coating 23, which is not illustrated in FIG. 1, may serve to further protect the therapeutic 22 during both the positioning and use of the medical device 20. The coating 23 may be a polymer, among other things, and may serve to shield the therapeutic during delivery and placement of the medical device 20. Once positioned at a target delivery site, the coating 23 may then dissolve, exposing the therapeutic 22, which may then be driven off of the medical device 20 by the driving layer 21. The coating 23 may cover all of the therapeutic 22 as well as only portions of it and may be applied through the same techniques used to apply the therapeutic and the driving layer. Other application techniques may be used as well. When the medical device 20 is a stent to be delivered with a catheter, the coating 23 may also serve to protect the therapeutic on the stent during the crimping process. Once crimped, the stent may be maintained at −4° C. to further protect the coating and the other layers of material on the stent until their deployment.

FIG. 3 is a cross-sectional view of a stent 30 that has been coated with driving layers 31 and 34 and therapeutics 35 and 32. The driving layers and therapeutics may function and behave much like those discussed above. In this embodiment, however, rather than coating only a single side of a medical device, FIG. 3 illustrates that both sides of a medical device, in this case a stent 30, may be coated with a driving layer and therapeutic. By coating both the inside and the outside of the stent 30, vascular tissue that the stent contacts as well as fluids coursing through the vessel in which the stent will be implanted may be treated with the therapeutic and may benefit from the efficient delivery characteristics of the driving layer, especially when the driving layer is behaving as a transfection agent. While the therapeutic in this figure is not coated it a coating may be placed on either or both exposed faces of the therapeutic.

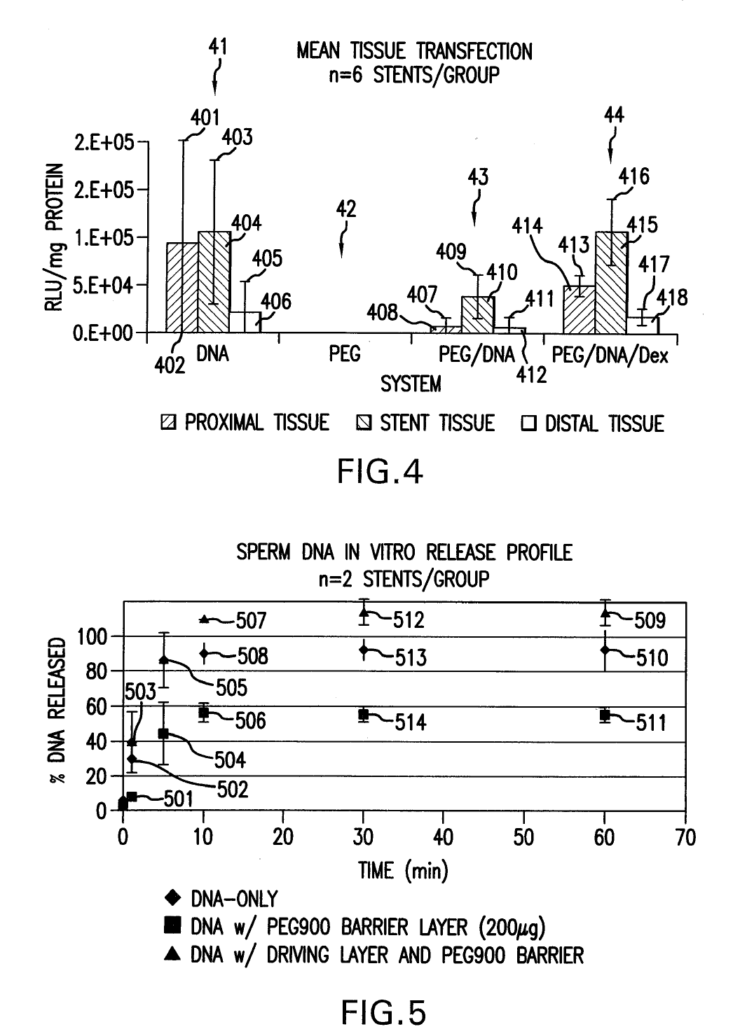

FIG. 4 is a table entitled Mean Tissue Transfection. It reflects the data from an in-vivo experiment using the invention, the mean of the delivered dose being shown with the height of the columns and the range of the delivered doses for each experiment being shown with the lines embedded within the columns. The first groups of columns 41 reflect that the range of delivered dose from a rabbit iliac study using a pCMV-Luc reporter with multiple 8 mm Express® WH stents coated only with DNA, delivered a broad range of dosages, from points 401-402, 403-404 and 405-406 for proximal tissue, stent tissue, and distal tissue, respectively. When the DNA was coated with PEG (columns 43), the range of dosage was found to be smaller (407-408, 409-410, 411-412), but the delivered dosage was also smaller. By comparison, when a driving layer of Dextran was used beneath the DNA and PEG coatings, see columns 44, not only was the range of dosage better controlled (413-414, 415-416, 417-418), but a greater dosage of DNA was delivered from the stent to the surrounding tissue when compared with the DNA/PEG stent control group (44 v. 43).

FIG. 5 shows the test results for a second experiment. The table in FIG. 5 is entitled, “Sperm DNA In Vitro Release Profile.” In this experiment, a DNA covered stent was compared with a stent covered with DNA and a PEG barrier coating and a stent covered with DNA, a driving layer, and a barrier coating. Almost without exception, the stent that utilized the driving layer and the barrier coating, labeled 505, 507, 509, 512, delivered the largest dosage at each time interval. Thus, even the DNA only covered stent which is depicted with numbers 502, 508, 510, and 513, did not exceed the delivery characteristics of the DNA/PEG/driving layer stent. Also, while not reflected in this table, which only reflects data from the first 70 minutes, the release of the DNA from the PEG only coated stents (501, 504, 506, 514, 511) was incomplete after the first day. Consequently, the addition of a Dextran driving layer in this experiment demonstrates the improved release properties of DNA from the carrier stents.

The therapeutic recited above may be any pharmaceutically acceptable agent such as a non-genetic therapeutic agent, a biomolecule, a small molecule, or cells. Exemplary non-genetic therapeutic agents include anti-thrombogenic agents such heparin, heparin derivatives, prostaglandin (including micellar prostaglandin E1), urokinase, and PPack (dextrophenylalanine proline arginine chloromethylketone); anti-proliferative agents such as enoxaprin, angiopeptin, sirolimus (rapamycin), tacrolimus, everolimus, monoclonal antibodies capable of blocking smooth muscle cell proliferation, hirudin, and acetylsalicylic acid; anti-inflammatory agents such as triamcinolone and derivatives, dexamethasone, rosiglitazone, prednisolone, corticosterone, budesonide, estrogen, estrodiol, sulfasalazine, acetylsalicylic acid, mycophenolic acid, and mesalamine; anti-neoplastic/anti-proliferative/anti-mitotic agents such as paclitaxel, cladribine, 5-fluorouracil, methotrexate, doxorubicin, daunorubicin, cyclosporine, cisplatin, vinblastine, vincristine, epothilones, endostatin, trapidil, and angiostatin; anti-cancer agents such as antisense inhibitors of c-myc oncogene; anti-microbial agents such as triclosan, cephalosporins, aminoglycosides, nitrofurantoin, silver ions, compounds, or salts; biofilm synthesis inhibitors such as non-steroidal anti-inflammatory agents and chelating agents such as ethylenediaminetetraacetic acid, O,O′-bis(2-aminoethyl)ethyleneglycol-N,N,N′,N′-tetraacetic acid and mixtures thereof, antibiotics such as gentamycin, rifampin, minocyclin, and ciprofloxacin; antibodies including chimeric antibodies and antibody fragments; anesthetic agents such as lidocaine, bupivacaine, and ropivacaine; nitric oxide; nitric oxide (NO) donors such as lisidomine, molsidomine, L-arginine, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, Warfarin sodium, Dicumarol, aspirin, prostaglandin inhibitors, platelet inhibitors and tick antiplatelet factors; vascular cell growth promoters such as growth factors, transcriptional activators, and translational promoters; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogenous vasoactive mechanisms; and any combinations and products of the above.

Exemplary biomolecules include peptides, polypeptides and proteins; oligonucleotides; nucleic acids such as double or single stranded DNA (including naked and cDNA), RNA, antisense nucleic acids such as antisense DNA and RNA, small interfering RNA (siRNA), and ribozymes; genes; carbohydrates; angiogenic factors including growth factors; cell cycle inhibitors; and anti-restenosis agents. Nucleic acids may be incorporated into delivery systems such as, for example, vectors (including viral vectors), plasmids or liposomes.

Non-limiting examples of proteins include monocyte chemoattractant proteins (MCP-1) and bone morphogenic proteins (BMP's), such as, for example, BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15. Preferred BMPS are any of BMP-2, BMP-3, BMP-4, BMP-5, BMP-6, and BMP-7. These BMPs can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNA's encoding them. Non-limiting examples of genes include survival genes that protect against cell death, such as anti-apoptotic Bcl-2 family factors and Akt kinase and combinations thereof. Non-limiting examples of angiogenic factors include acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor, and insulin like growth factor. A non-limiting example of a cell cycle inhibitor is a cathespin D (CD) inhibitor. Non-limiting examples of anti-restenosis agents include p15, p16, p18, p19, p21, p27, p53, p57, Rb, nFkB and E2F decoys, thymidine kinase (TK) and combinations thereof and other agents useful for interfering with cell proliferation.

Exemplary small molecules include hormones, nucleotides, amino acids, sugars, and lipids and compounds have a molecular weight of less than 100 kD.

Exemplary cells include stem cells, progenitor cells, endothelial cells, adult cardiomyocytes, and smooth muscle cells. Cells can be of human origin (autologous or allogenic) or from an animal source (xenogenic), or genetically engineered.

Any of the therapeutic agents may be combined to the extent such combination is biologically compatible.

With respect to the type of polymers that may be used in the coating according to the present invention, such polymers may be biodegradable or non-biodegradable. Non-limiting examples of suitable non-biodegradable polymers include polyvinylpyrrolidone including cross-linked polyvinylpyrrolidone; polyvinyl alcohols, copolymers of vinyl monomers such as EVA; polyvinyl ethers; polyvinyl aromatics; polyethylene oxides; polyesters including polyethylene terephthalate; polyamides; polyacrylamides; polyethers including polyether sulfone; polyalkylenes including polypropylene, polyethylene and high molecular weight polyethylene; polyurethanes; polycarbonates, silicones; siloxane polymers; cellulosic polymers such as cellulose acetate; polymer dispersions such as polyurethane dispersions (BAYHYDROL®); squalene emulsions; and mixtures and copolymers of any of the foregoing.

Non-limiting examples of suitable biodegradable polymers include polycarboxylic acid, polyanhydrides including maleic anhydride polymers; polyorthoesters; poly-amino acids; polyethylene oxide; polyphosphazenes; polylactic acid, polyglycolic acid and copolymers and mixtures thereof such as poly(L-lactic acid) (PLLA), poly(D,L-lactide), poly(lactic acid-co-glycolic acid), 50/50 (DL-lactide-co-glycolide); polydioxanone; polypropylene fumarate; polydepsipeptides; polycaprolactone and co-polymers and mixtures thereof such as poly(D,L-lactide-co-caprolactone) and polycaprolactone co-butylacrylate; polyhydroxybutyrate valerate and blends; polycarbonates such as tyrosine-derived polycarbonates and arylates, polyiminocarbonates, and polydimethyltrimethylcarbonates; cyanoacrylate; calcium phosphates; polyglycosaminoglycans; macromolecules such as polysaccharides (including hyaluronic acid; cellulose, and hydroxypropylmethyl cellulose; gelatin; starches; dextrans; alginates and derivatives thereof), proteins and polypeptides; and mixtures and copolymers of any of the foregoing. The biodegradable polymer may also be a surface erodible polymer such as polyhydroxybutyrate and its copolymers, polycaprolactone, polyanhydrides (both crystalline and amorphous), maleic anhydride copolymers, and zinc-calcium phosphate.

In a preferred embodiment, the polymer is polyacrylic acid available as HYDROPLUS® (Boston Scientific Corporation, Natick, Mass.), and described in U.S. Pat. No. 5,091,205, the disclosure of which is incorporated by reference herein. In a more preferred embodiment, the polymer is a co-polymer of polylactic acid and polycaprolactone.

Such coatings used with the present invention may be formed by any method known to one in the art. For example, an initial polymer/solvent mixture can be formed and then the therapeutic agent added to the polymer/solvent mixture. Alternatively, the polymer, solvent, and therapeutic agent can be added simultaneously to form the mixture. The polymer/solvent mixture may be a dispersion, suspension or a solution. The therapeutic agent may also be mixed with the polymer in the absence of a solvent. The therapeutic agent may be dissolved in the polymer/solvent mixture or in the polymer to be in a true solution with the mixture or polymer, dispersed into fine or micronized particles in the mixture or polymer, suspended in the mixture or polymer based on its solubility profile, or combined with micelle-forming compounds such as surfactants or adsorbed onto small carrier particles to create a suspension in the mixture or polymer. The coating may comprise multiple polymers and/or multiple therapeutic agents.

As recited above, the coating can be applied to the medical device by any known method in the art including dipping, spraying, rolling, brushing, electrostatic plating or spinning, vapor deposition, air spraying including atomized spray coating, and spray coating using an ultrasonic nozzle.

The coating is typically from about 1 to about 50 microns thick. In the case of balloon catheters, the thickness is preferably from about 1 to about 10 microns, and more preferably from about 2 to about 5 microns. Very thin polymer coatings, such as about 0.2-0.3 microns and much thicker coatings, such as more than 10 microns, are also possible. It is also within the scope of the present invention to apply multiple layers of polymer coatings onto the medical device. Such multiple layers may contain the same or different therapeutic agents and/or the same or different polymers.

The medical device may also contain a radio-opacifying agent within its structure to facilitate viewing the medical device during insertion and at any point while the device is implanted. Non-limiting examples of radio-opacifying agents are bismuth subcarbonate, bismuth oxychloride, bismuth trioxide, barium sulfate, tungsten, and mixtures thereof.

Non-limiting examples of medical devices according to the present invention include catheters, guide wires, balloons, filters (e.g., vena-cava filters), stents, stent grafts, vascular grafts, intraluminal paving systems, implants and other devices used in connection with drug-loaded polymer coatings. Such medical devices may be implanted or otherwise utilized in body lumina and organs such as the coronary vasculature, esophagus, trachea, colon, biliary tract, urinary tract, prostate, brain, and the like.

In addition to the various teachings provided above, other examples of the present invention are also possible. For instance, the thicknesses of the various layers may be varied without straying from the teachings of this disclosure. Moreover, portions of the medical implant may contain a barrier/driving layer and a therapeutic while other portions may contain these two in addition to a coating over the therapeutic. Still further, multiple layers of therapeutic or the driving layer may also be used.

What is claimed is:

1. A medical device comprising:
a second coating layer disposed over a surface of the medical device, wherein the second coating layer comprises a therapeutic agent; and
a first coating layer disposed between the surface of the medical device and the second coating layer, wherein the first coating layer comprises a material that enhances the delivery of the therapeutic agent to body tissue.

2. The medical device of claim 1, wherein the first coating layer is a driving layer having a driving material that promotes the release of the therapeutic agent from the second coating layer, and wherein the driving material has a higher aqueous solubility than the therapeutic agent.

3. The medical device of claim 1, wherein the first coating layer is a driving layer having a driving material that promotes the release of the therapeutic agent from the second coating layer, and wherein the driving material is biodegradable in body fluid or tissue.

4. The medical device of claim 2, wherein the surface of the medical device is hydrophobic and the therapeutic agent is hydrophilic.

5. The medical device of claim 2, further comprising a third coating layer disposed over at least a portion of the second coating layer.

6. The medical device of claim 2, wherein the driving layer also serves to retain the second coating layer on the medical device.

7. The medical device of claim 2, wherein the driving layer is a protein and the therapeutic agent is DNA.

8. The medical device of claim 2, wherein the medical device is a stent.

9. The medical device of claim 2, wherein the therapeutic agent is DNA and wherein the driving material is a transfection agent.

10. The medical device of claim 2, wherein the driving material is selected from the group consisting of: an ionic salt, an ionic surfactant, a non-ionic surfactant, a lipid, a polysaccharide, Dextran, a foaming agent, and a block co-polymer.

11. The medical device of claim 1, wherein the medical device is a bone implant.

12. The medical device of claim 2, wherein the driving layer effects the complete release of the therapeutic agent from the second coating layer when the medical device is at the target site.

13. A method for delivering a therapeutic agent to a body tissue, comprising:
providing a medical device comprising:
(a) a second coating layer disposed over a surface of the medical device, wherein the second coating layer comprises a therapeutic agent; and
(b) a first coating layer disposed between the surface of the medical device and the second coating layer, wherein the first coating layer comprises a material that enhances the delivery of the therapeutic agent to body tissue; and
delivering the medical device to the body tissue.

14. The method of claim 13, wherein the first coating layer is a driving layer having a driving material that promotes the release of the therapeutic agent from the second coating layer, and wherein the driving material has a higher aqueous solubility than the therapeutic agent.

15. The method of claim 14, further comprising implanting the medical device in the body tissue.

16. The method of claim 14, wherein the driving material promotes the release of the therapeutic agent from the second coating layer, and wherein the driving material is biodegradable in body fluid or tissue.

17. The method of claim 14, wherein the surface of the medical device is hydrophobic and the therapeutic agent is hydrophilic.

18. The method of claim 14, further comprising a third coating layer disposed over at least a portion of the second coating layer.

19. The method of claim 14, wherein the therapeutic agent is DNA and wherein the driving material is a transfection agent.

20. The method of claim 14, wherein the driving material is selected from the group consisting of: an ionic salt, an ionic surfactant, a non-ionic surfactant, a lipid, a polysaccharide, Dextran, a foaming agent, and a block co-polymer.
